(19) United States
(12) Patent Application Publication
(10) Pub. No.: US 2013/0066436 A1
(43) Pub. Date: Mar. 14, 2013

(54) Implants and Methods of Making and Using the Same

(71) Applicant: Jonathan Desa, Savannah, GA (US)

(72) Inventor: Jonathan Desa, Savannah, GA (US)

(21) Appl. No.: 13/606,758

(22) Filed: Sep. 7, 2012

Related U.S. Application Data
(60) Provisional application No. 61/573,520, filed on Sep. 8, 2011.

(51) Int. Cl. A61F 2/42; B23P 17/04

(52) U.S. Cl. 623/21.17; 29/592; 29/428

(57) ABSTRACT

Implants are disclosed. Methods of making and using implants are also disclosed.

### CROSS-REFERENCE TO RELATED APPLICATIONS

This patent application claims the benefit of priority to U.S. Provisional Patent Application Ser. No. 61/573,520 filed on Sep. 8, 2011 and entitled “PROXIMAL INTERPHALANGEAL (PIP) ENDOPROSTHESIS (IMPLANT)”, the subject matter of which is hereby incorporated by reference in its entirety.

### FIELD OF THE INVENTION

The present invention relates to implants and methods of making and using implants.

### BACKGROUND OF THE INVENTION

Proximal interphalangeal (PIP) joint deformities occur through congenital defects, trauma, or degenerative disease, in particular, rheumatoid arthritis. Rheumatoid arthritis is chronic inflammatory disorder in joints and leads to cartilage deterioration over time due to pH imbalances. Once the cartilage is destroyed, the joint loses the ability to move smoothly due to bone on bone contact. This in turn causes patient pain. To alleviate pain due to joint damage, many patients chose to undergo an arthroplasty (i.e., joint replacement) surgery.

Currently, the Swanson endoprosthesis implant is the primary choice of surgeons to perform an arthroplasty of the PIP joint. The Swanson implant is made from silicone rubber and undergoes high cyclic loading during normal patient usage. Over time, the Swanson implant leads to material failure and joint dislocation.

There exists a need in the art for improved implants that provide years of normal patient use and minimize the likelihood of material failure.

### SUMMARY OF THE INVENTION

In order to eliminate material failure and joint dislocation in a high cyclical loading environment, a novel endoprosthesis (implant) design has been developed, for example, as a replacement joint/implant for the PIP joint. The implants of the present invention may comprise (or consist essentially of or consist of) one or more components that enable the implant to be surgically implanted with minimal trauma to an existing joint. Further, the implants of the present invention may comprise (or consist essentially of or consist of) one or more components that enable long implant life while providing a relatively high degree of joint movement during use.

In one exemplary embodiment, the implant of the present invention comprises (I) a proximal component comprising a base shell member having a shell inner surface, a shell outer surface opposite the shell inner surface, a base shell member thickness separating the shell inner surface from the shell outer surface, a shell member leading end extending between the shell inner surface and the shell outer surface, a shell member trailing end extending between the shell inner surface and the shell outer surface and being opposite the shell member leading end, and a shell arch of curvature extending along the shell inner surface, the shell arch of curvature having a shell arch angle of greater than 180°; and a proximal component stem extending outwardly from the shell outer surface of the base shell member; (II) a distal component comprising a base head member having a head outer surface, two opposite head side surfaces separated from one another by the head outer surface, and a head arch of curvature extending along the head outer surface, the head arch of curvature having a head arch angle of 360°, and the base head member being sized and operatively adapted to be positioned within the base shell member; and a distal component stem extending outwardly from the head outer surface of the base head member; and (III) a cup component sized and operatively adapted to be positioned between (i) the shell inner surface of the proximal component and (ii) the head outer surface of the distal component, the cup component having a cup inner surface, a cup outer surface opposite the cup inner surface, a cup thickness separating the cup inner surface from the cup outer surface, a cup leading end extending between the cup inner surface and the cup outer surface, a cup trailing end extending between the cup inner surface and the cup outer surface and being opposite the cup leading end, and a cup arch of curvature extending along the cup inner surface, the cup arch of curvature having a cup arch angle of greater than 180°.

In some embodiments, the implants of the present invention comprise an articulating titanium alloy head component on an ultra high molecular weight polyethylene cup component, which is backed by a titanium alloy shell component as described herein. Proximal and distal stems of the disclosed implants may be used to fixate the head component and cup system to bones in a given joint, such as the PIP joint.

The present invention is further directed to methods of making any of the herein-disclosed implants. In one exemplary embodiment, the method of making an implant of the present invention comprises (I) forming a proximal component comprising a base shell member having a shell inner surface, a shell outer surface opposite the shell inner surface, a base shell member thickness separating the shell inner surface from the shell outer surface, a shell member leading end extending between the shell inner surface and the shell outer surface, a shell member trailing end extending between the shell inner surface and the shell outer surface and being opposite the shell member leading end, and a shell arch of curvature extending along the shell inner surface, the shell arch of curvature having a shell arch angle of greater than 180°; and a proximal component stem extending outwardly from the shell outer surface of the base shell member; (II) forming a distal component comprising a base head member having a head outer surface, two opposite head side surfaces separated from one another by the head outer surface, and a head arch of curvature extending along the head outer surface, the head arch of curvature having a head arch angle of 360°, and the base head member being sized and operatively adapted to be positioned within the base shell member; and a distal component stem extending outwardly from the head outer surface of the base head member; and (III) forming a cup component sized and operatively adapted to be positioned between (i) the shell inner surface of the proximal component and (ii) the head outer surface of the distal component, the cup component having a cup inner surface, a cup outer surface opposite the cup inner surface, a cup thickness separating the cup inner surface from the cup outer surface, a cup leading end extending between the cup inner surface and the cup outer surface, a cup trailing end extending between the cup inner surface and the cup outer surface and being opposite the cup leading end, and a cup arch of curvature extending along the cup inner surface, the cup arch of curvature having a cup arch angle of greater than 180°.

The methods of making any of the herein-disclosed implants of the present invention may further comprise one or more additional steps including, but not limited to, assembling the proximal component, the distal component and the cup component with one another so that (i) the cup component is positioned within the proximal component so that the cup outer surface extends along the shell inner surface, and (ii) the distal component is positioned within the cup component so that the head outer surface extends along the cup inner surface.

The present invention is even further directed to methods of using any of the herein-disclosed implants. In one exemplary embodiment, the method of using an implant of the present invention comprises implanting the implant into a patient, wherein the implant comprises(I) a proximal component comprising a base shell member having a shell inner surface, a shell outer surface opposite the shell inner surface, a base shell member thickness separating the shell inner surface from the shell outer surface, a shell member leading end extending between the shell inner surface and the shell outer surface, a shell member trailing end extending between the shell inner surface and the shell outer surface and being opposite the shell member leading end, and a shell arch of curvature extending along the shell inner surface, the shell arch of curvature having a shell arch angle of greater than 180°; and a proximal component stem extending outwardly from the shell outer surface of the base shell member; (II) a distal component comprising a base head member having a head outer surface, two opposite head side surfaces separated from one another by the head outer surface, and a head arch of curvature extending along the head outer surface, the head arch of curvature having a head arch angle of 360°, and the base head member being sized and operatively adapted to be positioned within the base shell member; and a distal component stem extending outwardly from the head outer surface of the base head member; and (III) a cup component sized and operatively adapted to be positioned between (i) the shell inner surface of the proximal component and (ii) the head outer surface of the distal component, the cup component having a cup inner surface, a cup outer surface opposite the cup inner surface, a cup thickness separating the cup inner surface from the cup outer surface, a cup leading end extending between the cup inner surface and the cup outer surface, a cup trailing end extending between the cup inner surface and the cup outer surface and being opposite the cup leading end, and a cup arch of curvature extending along the cup inner surface, the cup arch of curvature having a cup arch angle of greater than 180°.

The methods of using any of the herein-disclosed implants of the present invention may further comprise one or more additional steps including, but not limited to, attaching the proximal component stem to a first bone; and attaching the distal component stem to a second bone. In some desired embodiments, the implant of the present invention is used to replace a proximal interphalangeal (PIP) joint of the patient.

These and other features and advantages of the present invention will become apparent after a review of the following detailed description of the disclosed embodiments and the appended claims.

### BRIEF DESCRIPTION OF THE FIGURES

The present invention is further described with reference to the appended figures showing exemplary embodiments of the present invention, wherein:

FIG. 1 depicts a side view of an exemplary implant of the present invention;

FIGS. 2A-2C depict various views of an exemplary proximal component suitable for use in the exemplary implant shown in FIG. 1;

FIGS. 3A-3B depict various views of an exemplary distal component suitable for use in the exemplary implant shown in FIG. 1;

FIGS. 4A-4C depict various views of an exemplary cup component suitable for use in the exemplary implant shown in FIG. 1; and

FIG. 5 depicts a perspective view of the exemplary implant shown in FIG. 1 positioned within a finger of a patient.

### DETAILED DESCRIPTION OF THE INVENTION

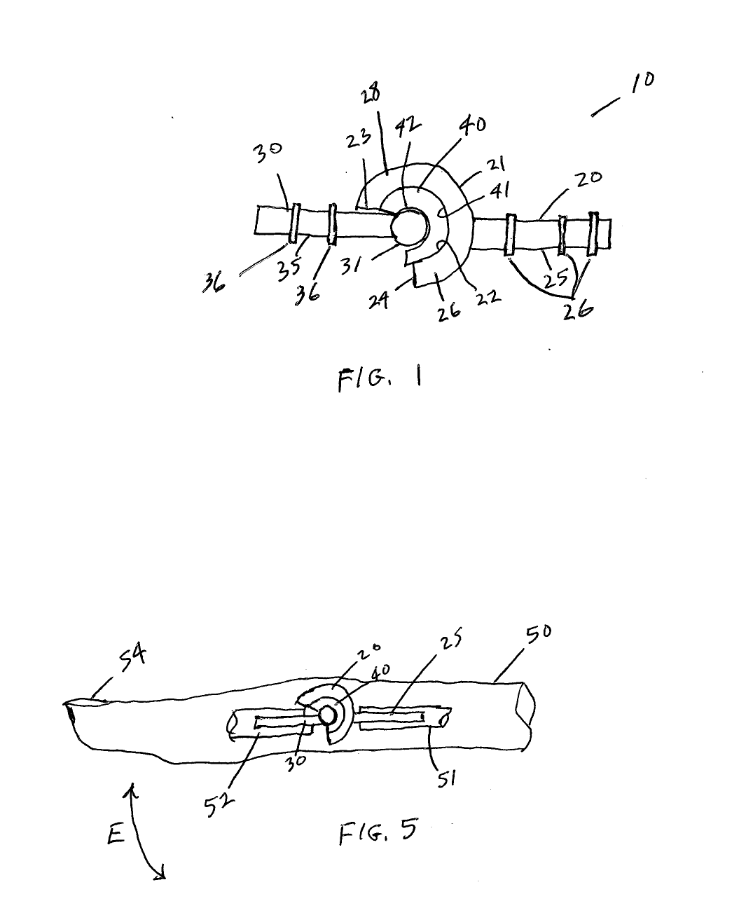

The present invention is directed to implants and methods of making and using implants. In particular, the present invention is directed to implants as shown in the figures, as described in the various embodiments shown and described herein, and as recited in the claims below. An exemplary implant 10 of the present invention is shown in FIG. 1.

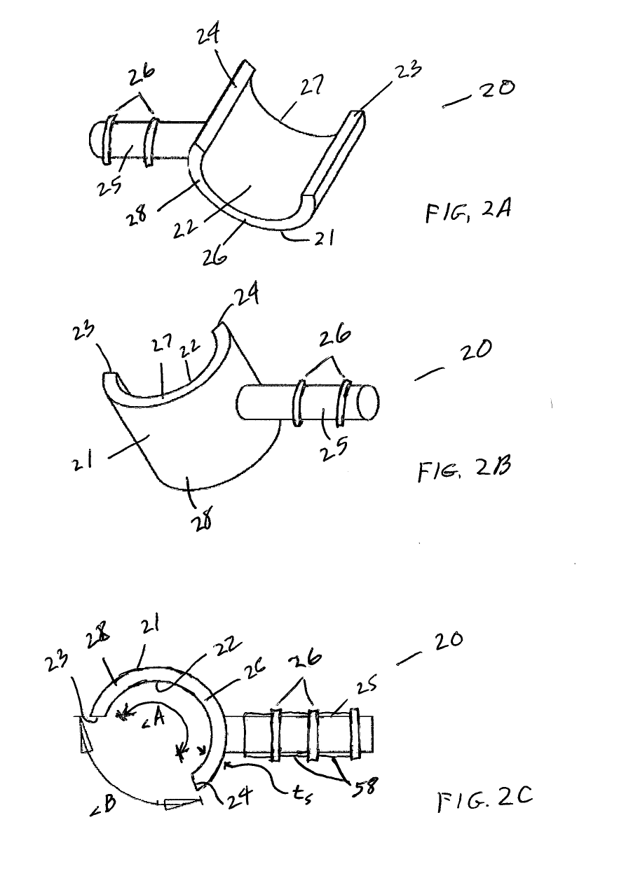

As shown in FIG. 1, exemplary implant 10 comprises an exemplary proximal component 20, an exemplary distal component 30, and an exemplary cup component 40. As further shown in FIGS. 1 and 2A-2C, exemplary proximal component 20 comprises a base shell member 28 having a shell inner surface 22, a shell outer surface 21 opposite shell inner surface 22, and a base shell member thickness, tₛ, separating shell inner surface 22 from shell outer surface 21. Base shell member 28 further comprises a shell member leading end 23 extending between shell inner surface 22 and shell outer surface 21, a shell member trailing end 24 extending between shell inner surface 22 and shell outer surface 21 and being opposite shell member leading end 23, and a shell arch of curvature extending along shell inner surface 22, the shell arch of curvature having a shell arch angle A of greater than 180°. Base shell member 28 further comprises a proximal component stem 25 extending outwardly from shell outer surface 21 of base shell member 28.

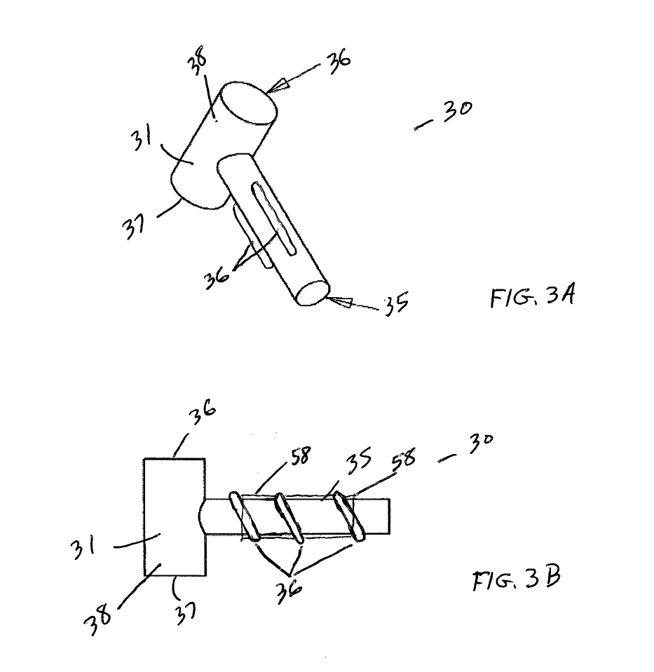

As shown in FIGS. 1 and 3A-3B, exemplary distal component 30 comprises a base head member 38 having a head outer surface 31, and two opposite head side surfaces 36 and 37 separated from one another by head outer surface 31. Base head member 38 has a head arch of curvature extending along head outer surface 31, wherein the head arch of curvature has a head arch angle of 360°. As shown in FIG. 1, base head member 38 is sized and operatively adapted to be positioned within base shell member 28. Exemplary distal component 30 further comprises distal component stem 35 extending outwardly from head outer surface 31 of base head member 38.

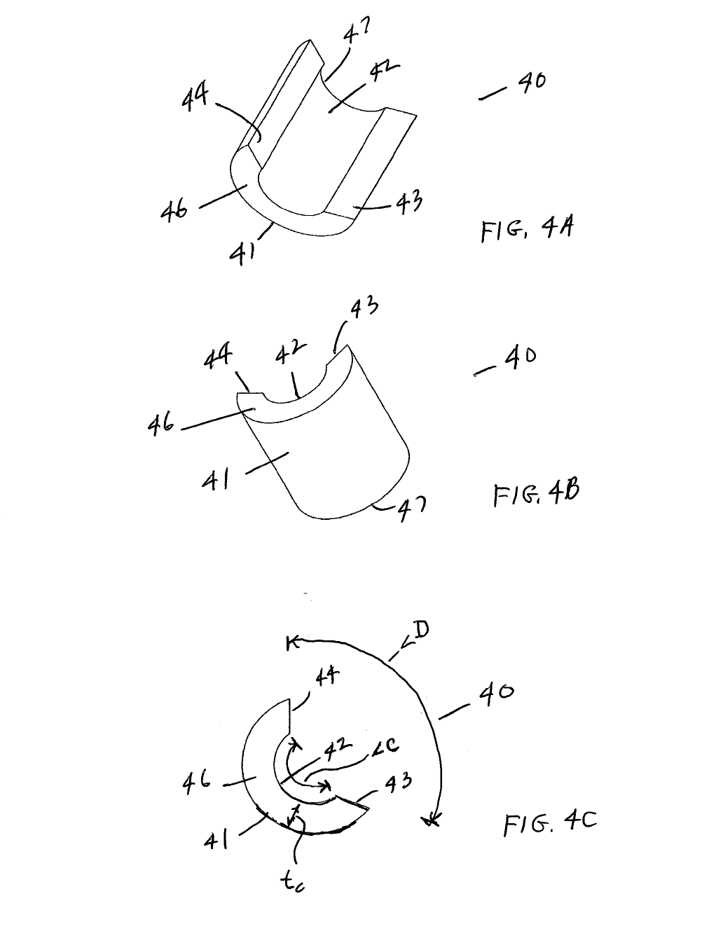

As shown in FIGS. 1 and 4A-4C, exemplary cup component 40 is sized and operatively adapted to be positioned between (i) shell inner surface 22 of proximal component 20 and (ii) head outer surface 31 of distal component 30. Exemplary cup component 40 has a cup inner surface 42, a cup outer surface 41 opposite cup inner surface 42, and a cup thickness, t_{c}, separating cup inner surface 42 from cup outer surface 41. Exemplary cup component 40 further comprises a cup leading end 43 extending between cup inner surface 42 and cup outer surface 41, a cup trailing end 44 extending between cup inner surface 42 and cup outer surface 41 and being opposite cup leading end 43. Exemplary cup component 40 further comprises a cup arch of curvature extending along cup inner surface 42, wherein the cup arch of curvature has a cup arch angle C of greater than 180°.

### Other Embodiments

Implants:

1. An implant 10 comprising: (I) a proximal component 20 comprising (a) a base shell member 28 having a shell inner surface 22, a shell outer surface 21 opposite said shell inner surface 22, a base shell member thickness, tₛ, separating said shell inner surface 22 from said shell outer surface 21, a shell member leading end 23 extending between said shell inner surface 22 and said shell outer surface 21, a shell member trailing end 24 extending between said shell inner surface 22 and said shell outer surface 21 and being opposite said shell member leading end 23, and a shell arch of curvature extending along said shell inner surface 22, said shell arch of curvature having a shell arch angle A of greater than 180°; and (b) a proximal component stem 25 extending outwardly from said shell outer surface 21 of said base shell member 28; (II) a distal component 30 comprising (a) a base head member 38 having a head outer surface 31, two opposite head side surfaces 36 and 37 separated from one another by said head outer surface 31, and a head arch of curvature extending along said head outer surface 31, said head arch of curvature having a head arch angle of 360°, said base head member 38 being sized and operatively adapted to be positioned within said base shell member 28; and (b) a distal component stem 35 extending outwardly from said head outer surface 31 of said base head member 38; and (III) a cup component 40 sized and operatively adapted to be positioned between (i) said shell inner surface 22 of said proximal component 20 and (ii) said head outer surface 31 of said distal component 30, said cup component 40 having a cup inner surface 42, a cup outer surface 41 opposite said cup inner surface 42, a cup thickness, t_{c}, separating said cup inner surface 42 from said cup outer surface 41, a cup leading end 43 extending between said cup inner surface 42 and said cup outer surface 41, a cup trailing end 44 extending between said cup inner surface 42 and said cup outer surface 41 and being opposite said cup leading end 43, and a cup arch of curvature extending along said cup inner surface 42, said cup arch of curvature having a cup arch angle C of greater than 180°. Cup arch angle C (as shown in FIG. 4C) may be any angle value from about 180° to about 300°, or within any range of angle values therebetween, e.g., from about 240° to about 260°, in increments of 1.0°. Further, as shown in FIG. 4C, angle D may comprise any angle less than about 180°, and desirably, any angle value from about 80° to about 180°, or within any range of angle values therebetween, e.g., from about 90° to about 110°, in increments of 1.0°.

2. The implant of embodiment 1, wherein said shell arch of curvature comprises a shell arch angle A of from about 190° to about 280°. Shell arch angle A (as shown in FIG. 2C) may be any angle value greater than 180°, typically, from greater than 180° to about 300°, or within any range of angle values therebetween, e.g., from about 240° to about 260°, in increments of 1.0°. Further, as shown in FIG. 2C, angle B may comprise any angle less than about 180°, and desirably, any angle value from about 80° to about 180°, or within any range of angle values therebetween, e.g., from about 90° to about 110°, in increments of 1.0°.

3. The implant of embodiment 1 or 2, wherein said shell arch of curvature comprises a shell arch angle A of about 250°.

4. The implant of any one of embodiments 1 to 3, wherein said proximal component 20 has a base shell member 28 thickness, tₛ, of from about 1.00 millimeters (mm) to about 1.50 mm. Base shell member 28 thickness, tₛ, may be any thickness value from about 1.00 mm to about 1.50 mm, or any range of thickness values therebetween, in increments of 0.01 mm.

5. The implant of any one of embodiments 1 to 4, wherein said proximal component stem 25 and said distal component stem 35 each independently extend a distance of at least about 10.0 mm from said shell outer surface 21 and said head outer surface 31, respectively. Typically, said proximal component stem 25 and said distal component stem 35 each independently extend a distance of less than about 20.0 mm, and more typically from about 12.0 mm to about 15.0 mm or any value or range of values therebetween, in increments of 0.01 mm. It should be noted that although stems 25 and 35 are shown as having a circular cross-sectional configuration, each of stems 25 and 35 may independently have any cross-sectional configuration. Suitable cross-sectional configurations include, but are not limited to, circular, oval, triangular, half-moon (i.e., half of a circle), rectangular, square, hexagonal, star-shaped, etc. cross-sectional configurations.

6. The implant of any one of embodiments 1 to 5, wherein said proximal component stem 25 and said distal component stem 35 each independently comprise ridges 26 and 36 extending along a length thereof. As shown in FIGS. 1-3B, optional ridges 26 and 36 may be present in one or multiple locations along stems 25 and 35, and may have a variety of configurations. For example, as shown in FIGS. 1-2C, optional ridges 26 and 36 may have a circular configuration extending along an outer periphery of stems 25 and 35, and spaced from one another along a length of stems 25 and 35. In other embodiments, as shown in FIGS. 3A-3B, optional ridges 26 and 36 may have a linear configuration extending along a length of stem 25 and/or stem 35 (i.e., see ridges 36 along stem 35 in FIG. 3A) or a circular angled configuration along a length of stem 25 and/or stem 35 (i.e., see ridges 36 along stem 35 in FIG. 3B). As shown in FIGS. 2C and 3B, stems 25 and 35 may further comprise a thin layer 58 (e.g., having a layer thickness of up to about 100 microns (μm) or any thickness greater than 0 up to 100 μm, in increments of 0.1 μm) of hydroxyapatite (e.g., beaded hydroxyapatite) along at least a portion thereof to promote bone re-growth after implantation.

7. The implant of any one of embodiments 1 to 6, wherein said base head member 28 has a base head member 28 diameter of about 5.0 mm. Base head member 28 may have a base head member 28 diameter of from about 3.0 mm to about 8.0 mm, or any value therebetween or within any range of values therebetween, e.g., from about 4.5 mm to about 5.5 mm, in increments of 0.01 mm.

8. The implant of any one of embodiments 1 to 7, wherein said cup arch of curvature comprises a cup arch angle C of from about 190° to about 280°. As discussed above, cup arch angle C (as shown in FIG. 4C) may be any angle value from about 180° to about 300°, or within any range of angle values therebetween, e.g., from about 240° to about 260°, in increments of 1.0°.

9. The implant of any one of embodiments 1 to 8, wherein said cup arch of curvature comprises a cup arch angle C of about 250°.

10. The implant of any one of embodiments 1 to 9, wherein said proximal component 20 and said distal component 30 each independently comprise a titanium alloy. Suitable titanium alloy components or other alloy components (e.g., other than titanium alloys) include, but are not limited to, titanium, aluminum, vanadium, iron, oxygen, cobalt, chromium, molybdenum, or any combination thereof. It should be noted that materials other than titanium alloys may be used to form proximal component 20 and distal component 30. For example, stainless steel could be used to form proximal component 20 and distal component 30.

11. The implant of any one of embodiments 1 to 10, wherein said cup component 40 comprise a polyethylene. Cup component 40 may comprise other materials including, but not limited to, ceramic, metal (e.g. gold, silver, cobalt-chromium alloys), polycarbonate, polyurethane, nylon and thermoplastic elastomers.

12. The implant of any one of embodiments 1 to 11, wherein said proximal component 20 and said distal component 30 each independently comprise a titanium alloy comprising Ti-6Al-4V, and said cup component 40 comprise a polyethylene having a weight average molecular weight of from about 3.0 to about 5.8 million (e.g., an ultra high molecular weight polyethylene, UHMWPE). The UHMWPE typically has a density ranging from about 0.93 to about 0.935 g/cm³.

13. The implant of any one of embodiments 1 to 12, wherein said cup component 40 is positioned within said proximal component 20 so that said cup outer surface 41 extends along said shell inner surface 22, and said distal component 30 is positioned within said cup component 40 so that said head outer surface 31 extends along said cup inner surface 42. It should be noted that the implants of the present invention may include additional components not shown. For example, in some embodiments, the implant (or a kit containing the implant) may further comprise one or more elastic bands (e.g., silicone-rubber bands) sized so as to (i) be positioned around and along outer surface 21 of proximal component 20 and (ii) encompass (e.g., surround) distal component 30 and cup component 40 positioned within proximal component 20. In this embodiment, outer surface 21 of proximal component 20 may be formed so as to have one or more grooves therein so as to house (e.g., contain) portions of one or more elastic bands surrounding proximal component 20, distal component 30 and cup component 40 and compressing proximal component 20, distal component 30 and cup component 40 with one another.

21. A kit comprising one or more implants 10 and/or one or more implant components (e.g., proximal components 20, distal components 30, cup components 40, elastic bands, hydroxyapatite, etc., or any combination thereof) as described in any of embodiments 1 to 13.

Methods of Making Implants:

14. A method of making the implant 10 of any one of embodiments 1 to 13, said method comprising: forming the proximal component 20; forming the distal component 30; and forming the cup component 40.

15. The method of embodiment 14, wherein each of said forming steps comprises a thermoforming step. Suitable thermoforming steps include, but are not limited to, a molding step, an injection molding step, a molding step in combination with a machining step, etc. The method steps of forming any one or each of proximal component 20, distal component 30, and cup component 40 may include, but are not limited to, one or more of the following method steps: coating any portion or all of the component with one or more coating materials (e.g., a lubricant to improve slippage/sliding between adjacent component surfaces, a material to improve bonding with a bone (e.g., hydroxyapatite), etc.); cutting proximal component stem 25 and/or distal component stem 35 to a desired length; etc.

16. The method of embodiment 14 or 15, further comprising: assembling the proximal component 20, the distal component 30 and the cup component 40 with one another so that (i) the cup component 40 is positioned within the proximal component 20 so that the cup outer surface 41 extends along the shell inner surface 22, and (ii) the distal component 30 is positioned within the cup component 40 so that the head outer surface 31 extends along the cup inner surface 42.

Methods of Using Implants:

17. A method of using the implant 10 of any one of embodiments 1 to 13, said method comprising: implanting the proximal component 20, the distal component 30 and the cup component 40 into a patient. The implanting step may comprise one or more of the following steps: making an incision in the patient, drilling a hole in a bone of the patient, inserting proximal component stem 25 and/or the distal component stem 35 into a hole within a bone of the patient, optionally applying a bonding agent onto stems 25 and/or 35 and/or within a hole within a bone of the patient, assembling the proximal component 20, the distal component 30 and the cup component 40 with one another so that (i) the cup component 40 is positioned within the proximal component 20 so that the cup outer surface 41 extends along the shell inner surface 22, and (ii) the distal component 30 is positioned within the cup component 40 so that the head outer surface 31 extends along the cup inner surface 42, and closing an incision within the patient (e.g., via stitches or sutures).

18. The method of embodiment 17, further comprising: attaching the proximal component stem 25 to a first bone 51 (see, FIG. 5) and attaching the distal component stem 35 to a second bone 52 (see, FIG. 5). In one embodiment of the present invention, first and second bones 51 and 52 may be within a patient's finger 50 having fingernail 54 as shown in FIG. 5. In other embodiments of the present invention, first and second bones 51 and 52 may be any other combination of bones meeting within a joint of a patient.

19. The method of embodiment 17 or 18, wherein the implant replaces a proximal interphalangeal (PIP) joint of the patient.

20. A method of using the implant 10 of any one of embodiments 1 to 13, said method comprising: replacing the cup component 40 of the implant 10 while the proximal component 20 and the distal component 30 are attached to first and second bones 51 and 52 of a patient.

### EXAMPLES

### Example 1

### Manufacture of Implants

Implants, similar to implant 10 shown in FIGS. 1-5, were prepared. Engineering analysis, SolidWorks™ 3-D modeling and finite element analysis were used to optimize and validate the benefits of the implant design. All results suggested that the implant 10 design would not have a material failure in high cyclical loading environments, while restoring the natural range of motion of a joint, such as the PIP joint.

### Example 2

### Implantation of an Implant Into a Cadaver

The following surgical procedure was utilized to implant an implant 10 of Example 1 into a cadaver. A vertical cut to the medial side of the PIP joint of the cadaver was made. Once the cut was made, the distal and proximal phalanges were bent to allow exposure of their articulating surfaces. The distal phalange was drilled and the articulating cylindrical head (e.g., distal component stem 35 of distal component 30) was implanted into the distal phalanges. The proximal phalange was drilled and the socket component (i.e., proximal stem 35 of proximal component 30) was implanted into the proximal phalange. The joint was completed by placing the articulating cylindrical head 38 of distal component 30 inside the cylindrical socket 28 of proximal component 20 with cup component 40 positioned therebetween.

After insertion, fixation, and testing, ligaments and tendons were reattached, and sutures were used to close the incision.

It should be understood that although the above-described implants, kits, and methods are described as “comprising” one or more components or steps, the above-described implants, kits, and methods may “comprise,” “consists of,” or “consist essentially of” any of the above-described components, features or steps of the implants, kits, and methods. Consequently, where the present invention, or a portion thereof, has been described with an open-ended term such as “comprising,” it should be readily understood that (unless otherwise stated) the description of the present invention, or the portion thereof, should also be interpreted to describe the present invention, or a portion thereof, using the terms “consisting essentially of” or “consisting of” or variations thereof as discussed below.

As used herein, the terms “comprises,” “comprising,” “includes,” “including,” “has,” “having,” “contains”, “containing,” “characterized by” or any other variation thereof, are intended to encompass a non-exclusive inclusion, subject to any limitation explicitly indicated otherwise, of the recited components. For example, an implant, a kit, and/or method that “comprises” a list of elements (e.g., components, features or steps) is not necessarily limited to only those elements (or components or steps), but may include other elements (or components or steps) not expressly listed or inherent to the implant, kit, and/or method.

As used herein, the transitional phrases “consists of” and “consisting of” exclude any element, step, or component not specified. For example, “consists of” or “consisting of” used in a claim would limit the claim to the components, materials or steps specifically recited in the claim except for impurities ordinarily associated therewith (i.e., impurities within a given component). When the phrase “consists of” or “consisting of” appears in a clause of the body of a claim, rather than immediately following the preamble, the phrase “consists of” or “consisting of” limits only the elements (or components or steps) set forth in that clause; other elements (or components) are not excluded from the claim as a whole.

As used herein, the transitional phrases “consists essentially of” and “consisting essentially of” are used to define an implant, a kit, and/or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

Further, it should be understood that the herein-described implants, kits, and methods may comprise, consist essentially of, or consist of any of the herein-described components and features, as shown in the figures with or without any feature(s) not shown in the figures. In other words, in some embodiments, the implants, kits, and/or methods of the present invention do not have any additional features other than those shown in the figures, and such additional features, not shown in the figures, are specifically excluded from the implants, kits, and/or methods. In other embodiments, the implants, kits, and/or methods of the present invention do have one or more additional features that are not shown in the figures.

While the specification has been described in detail with respect to specific embodiments thereof, it will be appreciated that those skilled in the art, upon attaining an understanding of the foregoing, may readily conceive of alterations to, variations of, and equivalents to these embodiments. Accordingly, the scope of the present invention should be assessed as that of the appended claims and any equivalents thereto.

What is claimed is:

1. An implant comprising:
a proximal component comprising:
a base shell member having a shell inner surface, a shell outer surface opposite said shell inner surface, a base shell member thickness separating said shell inner surface from said shell outer surface, a shell member leading end extending between said shell inner surface and said shell outer surface, a shell member trailing end extending between said shell inner surface and said shell outer surface and being opposite said shell member leading end, and a shell arch of curvature extending along said shell inner surface, said shell arch of curvature having a shell arch angle of greater than 180°; and
a proximal component stem extending outwardly from said shell outer surface of said base shell member;
a distal component comprising:
a base head member having a head outer surface, two opposite head side surfaces separated from one another by said head outer surface, and a head arch of curvature extending along said head outer surface, said head arch of curvature having a head arch angle of 360°, said base head member being sized and operatively adapted to be positioned within said base shell member; and
a distal component stem extending outwardly from said head outer surface of said base head member; and
a cup component sized and operatively adapted to be positioned between (i) said shell inner surface of said proximal component and (ii) said head outer surface of said distal component, said cup component having a cup inner surface, a cup outer surface opposite said cup inner surface, a cup thickness separating said cup inner surface from said cup outer surface, a cup leading end extending between said cup inner surface and said cup outer surface, a cup trailing end extending between said cup inner surface and said cup outer surface and being opposite said cup leading end, and a cup arch of curvature extending along said cup inner surface, said cup arch of curvature having a cup arch angle of greater than 180°.

2. The implant of claim 1, wherein said shell arch of curvature comprises a shell arch angle of from about 190° to about 280°.

3. The implant of claim 2, wherein said shell arch of curvature comprises a shell arch angle of about 250°.

4. The implant of claim 3, wherein said proximal component has a base shell member thickness of from about 1.00 millimeters (mm) to about 1.50 mm.

5. The implant of claim 1, wherein said proximal component stem and said distal component stem each independently extend a distance of at least about 10.0 mm from said shell outer surface and said head outer surface, respectively.

6. The implant of claim 5, wherein said proximal component stem and said distal component stem each independently comprise ridges extending along a length thereof.

7. The implant of claim 1, wherein said base head member has a base head member diameter of about 5.0 mm.

8. The implant of claim 1, wherein said cup arch of curvature comprises a cup arch angle of from about 190° to about 280°.

9. The implant of claim 8, wherein said cup arch of curvature comprises a cup arch angle of about 250°.

10. The implant of claim 1, wherein said proximal component and said distal component each independently comprise a titanium alloy.

11. The implant of claim 10, wherein said cup component comprise a polyethylene.

12. The implant of claim 1, wherein said proximal component and said distal component each independently comprise a titanium alloy comprising Ti-6Al-4V, and said cup component comprises a polyethylene.

13. The implant of claim 1, wherein said cup component is positioned within said proximal component so that said cup outer surface extends along said shell inner surface, and said distal component is positioned within said cup component so that said head outer surface extends along said cup inner surface.

14. A method of making the implant of claim 1, said method comprising:
forming the proximal component;
forming the distal component; and
forming the cup component.

15. The method of claim 14, wherein each of said forming steps comprises a thermoforming step.

16. The method of claim 15, further comprising:
assembling the proximal component, the distal component and the cup component with one another so that (i) the cup component is positioned within the proximal component so that the cup outer surface extends along the shell inner surface, and (ii) the distal component is positioned within the cup component so that the head outer surface extends along the cup inner surface.

17. A method of using the implant of claim 1, said method comprising:
implanting the proximal component, the distal component and the cup component into a patient.

18. The method of claim 17, further comprising:
attaching the proximal component stem to a first bone; and
attaching the distal component stem to a second bone.

19. The method of claim 18, wherein the implant replaces a proximal interphalangeal (PIP) joint of the patient.

20. A method of using the implant of claim 1, said method comprising:
replacing the cup component of the implant while the proximal component and the distal component are attached to first and second bones of a patient.
